(19) United States
(12) Patent Application Publication
(10) Pub. No.: US 2008/0034847 A1
(43) Pub. Date: Feb. 14, 2008

(54) Appartus and method for content discrimination

(76) Inventor: Lee B. Golter, Grand Junction, CO (US)

Correspondence Address: EDWIN L. HARTZ, 619 MAIN STREET, SUITE 115, GRAND JUNCTION, CO, 81501, US

(21) Appl. No.: 11/891,283

(22) Filed: Aug. 9, 2007

Related U.S. Application Data
(60) Provisional application No. 60/836,762, filed on Aug. 10, 2006.

(51) Int. Cl. G01N 33/00

(52) U.S. Cl. 73/61.41

(57) ABSTRACT

Liquid levels and transition levels of stratified liquids and gradients in one or more characteristics of liquids are detected by apparatus for and method of moving a sensor through the liquid.

### CROSS REFERENCE TO RELATED APPLICATION

This application claims priority to Provisional Application U.S. Application No. 60/836,762 filed Aug. 10, 2006.

### FIELD OF THE INVENTION

Apparatus and method for content discrimination and more particularly for the detection and use of surface levels and transition levels of stratified liquids and/or of the gradients in a liquid.

Content discrimination providing volume and relative position of different liquids in a container or different gradients in a physical, chemical or electrical property of a liquid is useful in many industries.

It is particularly useful in the gas industry where liquids are recovered from gas wells as a byproduct to the gas. These liquids called natural gas liquids (NGLs), are separated from the gas stream at the well site and placed in storage tanks (condensate tanks). Some of these same liquids are transported within the gas stream and are condensate out of the stream at compressor stations. The heavier liquids, BS&W and water primarily, are transported to water plants.

These liquids stratify in the storage tanks because of differences in specific gravity or density. Oil, being the lightest of the liquids, floats on top. There are various classes of oil related to the type and degree of contamination. The classes of oil from gas wells are identified and defined in a Bureau of Land Management (BLM) brochure entitled “Onshore Oil and Gas Operations; Federal and Indian Oil and Gas Leases; Onshore Oil and Gas Order No. 4; Measurement of Oil” issued under 43 CFR 3160 and published in the Federal Register at Volume 54, No. 36, Feb. 24, 1989 and effective Aug. 23, 1989. The upper liquids in order from the top are clean oil, dirty oil and waste oil. These liquids float on water with bottom sediment and water (BS&W) being at the bottom of the tank.

These liquids and the management of them by variable height inlet/outlet orifices are disclosed in PCT application Serial Number PCT/US2006/004479 filed Feb. 8, 2006 and U.S. Provisional Application No. ______ (Docket No. G13:1080P) filed May 16, 2007, both being assigned to the same Assignee as this application and disclosures thereof are incorporated in full herein by this reference as though set forth in full.

Some apparatus and methods of determining volume and position of the various NGLs are disclosed in Provisional Patent Application Ser. No. 60/810,013, filed May 31, 2006, the disclosure of this application is incorporated herein by this reference as though set forth in full.

Management of different liquids, and particularly the liquids from gas wells, is disclosed in the concurrently filed Provisional Patent Application having docket number G13:1070 and the disclosure in this application is incorporated herein by this reference as though set forth herein.

The invention disclosed and covered by the application is not limited to use in these particular liquids. However, for the purpose of understanding the concept and some applications, the invention will be disclosed in the context of liquids from natural gas wells stored in condensate tanks, such as the 20 foot high, 400-barrel or 500-barrel tanks used in gas fields. Typically there is air above the liquids, possibly saturated with gas vapors.

The content discrimination system of the invention provides the volume and levels of each stratified liquid. This information is available as numeric value, such as 50 barrels and between 12 and 14.48 feet high for a 400 barrel tank, or values that can be converted to volume and position or as a display indicating volume and position.

The information as to type of liquid is gathered by a capacitive sensor that is moved vertically through the various liquids. The capacitive sensor has capacitor plates that are surrounding liquids in the tank.

The capacitor plates may be rectangular in shape with facing parallel flat surfaces spaced a specific distance apart. Alternatively, the plates may be coaxial cylinders a selected distance apart or a single plate, with a nearby grounded body as the other plate.

The dielectric coefficient of air and each type of liquid results in a different capacitance as the air or liquid resides between the plates as the dielectric.

The capacitor plates may be electrically connected between an excitation terminal and an input terminal to digital converter. A particularly effective converter is the AD7745-24 Bit, 1 Channel Capacitance to Digital Converter from Analog Devices (www.analog.com). This Converter has an internal temperature compensation of the output of the circuit. An external temperature sensor, such as an RTD, thermister or diode for monitoring the temperature of each liquid or a liquid in a container, may be coupled to the Converter.

Another circuitry that may be used as the dielectric to digital converter consists of an astable oscillator and the capacitor plates in and insulated from the liquid. The capacitor plates are part of a capacitive circuit that involves a resistor to set the frequency of the oscillator. With air between the plates, the resistor is adjusted so that the frequency of the oscillator is 200 kilohertz. Thereafter, the frequency of the oscillator changes and represents the dielectric between the plates. The frequency can be calibrated with the different liquids in the tank so that the type of liquid is known from the frequency.

The operating frequency of the oscillator is converted to a digital signal by a microprocessor functioning as a signal processor. An external temperature sensor for sensing the temperature of the liquid between the plates also has an input to the microprocessor to appear as information at the output of the processor.

The capacitor plates of the oscillator for use in liquids from gas wells are 1 centimeter wide and 8 centimeters long with spacing between the plates of 1½ centimeters. The plates are covered by a thin layer of plastic, such as PVC, or some other material that will provide long term use in the environment of use, such as the corrosive liquids from gas wells. Also, the size and location of the plates will vary with the electronic circuitry coupled to the plates and the environment of use.

The dielectric to digital converter consisting of either the AD 7745 converter circuit, the astable oscillator and signal processor or some other circuitry having a digital signal output representative of the dielectric coefficient and thereby identifying each type of liquid in a tank. This digital signal out is applied to a communications protocol converter for use with any of the standard protocols used in the industry of interest.

The vertical movement of the content discrimination sensor in the liquids in a storage tank is monitored to provide the position or elevation of the sensor in the container and thus the detected levels of the liquids in the container. In one form of the invention the housing for the sensor is moved up and down by an adjusting and measuring rod that passes through a gear mechanism. The adjusting and measuring rod has teeth or indentions such as disclosed in the above-referenced PCT Application and also in the provisional patent application Ser. No. 60/810,013 filed May 31, 2006, the disclosure of which is incorporated herein by this reference as though set forth in full. The gear mechanism is driven by hand or by a motor. If driven by hand, the operator notes both the distance and direction of travel of the measuring rod and thus knows the position of the sensor inside the tank. If the adjusting and measuring rod is driven by a motor, a sensor for distance and direction of travel is included in the motor drive mechanism. Alternatively, the housing is attached to a nut on a vertical lead screw in the tank so the carriage moves vertically in the tank when the lead screw is turned. The nut and housing for the sensor are held in place to prevent rotation of the sensor by a guide or support pole that extends vertically in the tank adjacent to and parallel to the lead screw. The lead screw may also be turned manually or by a motor inside or outside the container. Additionally, instead of a separate carriage and drive mechanism for the movable sensor, the sensor may be carried on the same carriage attached to a nut on a lead screw as that for the housing for variable height inlet/outlet orifice device as disclosed in the above-referenced PCT Patent Application and as disclosed in the above-referenced provisional patent application filed on May 31, 2006.

The contents in a container such as a condensate tank at a well site, a compressor station, water plant or an injection well may be provided to instruments at the site, at the tank or at a remote site. The content discrimination system information may be used for inventory control, scheduling of trucks to reduce truck traffic, managing the liquids in the tank and/or managing a gas well.

The content discrimination probe disclosed in this application may be used as the volume and level sensor in the systems disclosed in the above-referenced Provisional Patent Application Ser. No. 60/810,013 filed May 31, 2006 and U.S. patent application Ser. No. 11/413,774 filed Apr. 28, 2006, the disclosure of this application is incorporated herein by this reference as though set forth in full.

The invention advantageously combines components and methodology of electronic automation with variable height inlet/outlet orifices and in-tank, variable-height content discrimination equipment to enable the accurate management of stratified layers in a liquid storage tank, or other similar type of tank in different industries, without the operator physically going to the top of the tank and adjusting the position of the inlet/outlet orifice to the correct height.

Additional features and advantages of the invention will be apparent from the detailed description which follows, taken in conjunction with the accompanying drawings, which together illustrate, by way of example, features of the invention; and wherein:

### BRIEF DESCRIPTION OF THE DRAWINGS

FIG. 1 is a block diagram of a content discrimination sensor, according to this invention;

FIG. 2 is a functional block diagram of a dielectric to digital converter, according to this invention;

FIG. 3 is a block diagram of an alternative dielectric to digital (frequency) converter, according to this invention;

FIG. 4 is an illustrative diagram of movable content discrimination sensor in a tank, according to this invention;

FIG. 5 is a sectional view of the moveable sensor in FIG. 4, according to this invention;

FIG. 6 is an elevation view of the moveable sensor of FIG. 4, according to this invention;

FIG. 7 in a top plan view of the moveable sensor of FIG. 4, according to this invention;

FIG. 8 is a photo of a test tank containing natural gas liquids and a content discrimination sensor, according to this invention; and

FIG. 9 is a graph of the output of the content discrimination sensor in the test tank of FIG. 8, according to this invention;

FIG. 10 is an illustrative diagram of a content discrimination sensor with a variable height inlet/outlet orifice tool, according to this invention;

FIG. 11 is a block diagram of a liquid management system, according to this invention;

FIG. 12 is a front-elevation view of a Bluetooth enabled, programmable PDA useful with the apparatus, in accordance with the present invention; and

FIG. 13 is a report available on a display or hard copy, in accordance with the present invention.

### DETAILED DESCRIPTION OF THE INVENTION

Reference will now be made to the exemplary embodiments illustrated in the drawings, and specific language will be used herein to describe the same. It will nevertheless be understood that no limitation of the scope of the invention is thereby intended. Alterations and further modifications of the inventive features illustrated herein, and additional applications of the principles of the inventions as illustrated herein, which would occur to one skilled in the relevant art and having possession of this disclosure, are to be considered within the scope of the invention.

Different physical, chemical and/or electrical characteristics of liquids in a tank can be sensed to determine the contents of the tank and when coupled with means for determining the position in the tank, the transitions from one liquid to another can also be determined. The different dielectric coefficients of liquids in a tank may be used to discriminate and identify the various liquids in a tank.

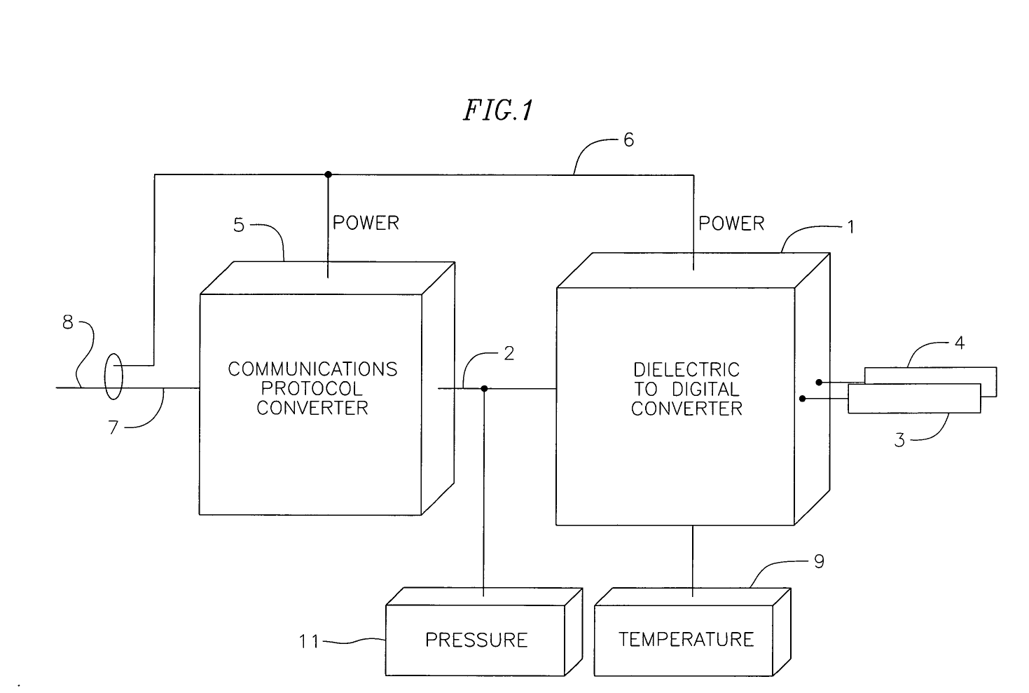

A capacitive probe is used to detect the coefficients of the liquids between the capacitive plates of the probe and creates an output signal indicative of the coefficient and therefore the type of liquid between the capacitor plates of the probe. The liquid or dielectric is identified and converted to a digital signal. A dielectric to digital converter creates a digital signal output which may be called the coefficient of the dielectric (liquid) in contact with the capacitor plates. Such a dielectric to digital converter 1 is shown in block form in FIG. 1. A pair of capacitor plates 3 and 4 is electrically connected to the dielectric to digital converter 1. The digital signal or converted coefficient that is the output of the converter 1 is applied as an input to a communication protocol converter 5 as shown in FIG. 1. Power is provided to both converters by a cable 6 that combines with the data output cable 7 from converter 5 as a power and data cable 8. The two converters 1 and 5 may advantageously be housed in the housing for the capacitance probe with the power and data cable communicating with circuitry outside of the tank in which the capacitive probe is located. The dielectric to digital 1 may also have an input from a temperature sensor such as an RTD, thermistor or diode. In this way, the temperature of the liquid or dielectric between the capacitor plates 3 and 4 may also monitored. Additionally, the converter 1 may have an input from a pressure sensor 11 that is useful in determining the volume of the liquid above the capacitive plates 3 and 4 when there is only one type of liquid above the capacitive probe 1 in the tank. The pressure is monitored by a pressure sensor 11 and the temperature is monitored by a temperature sensor 9.

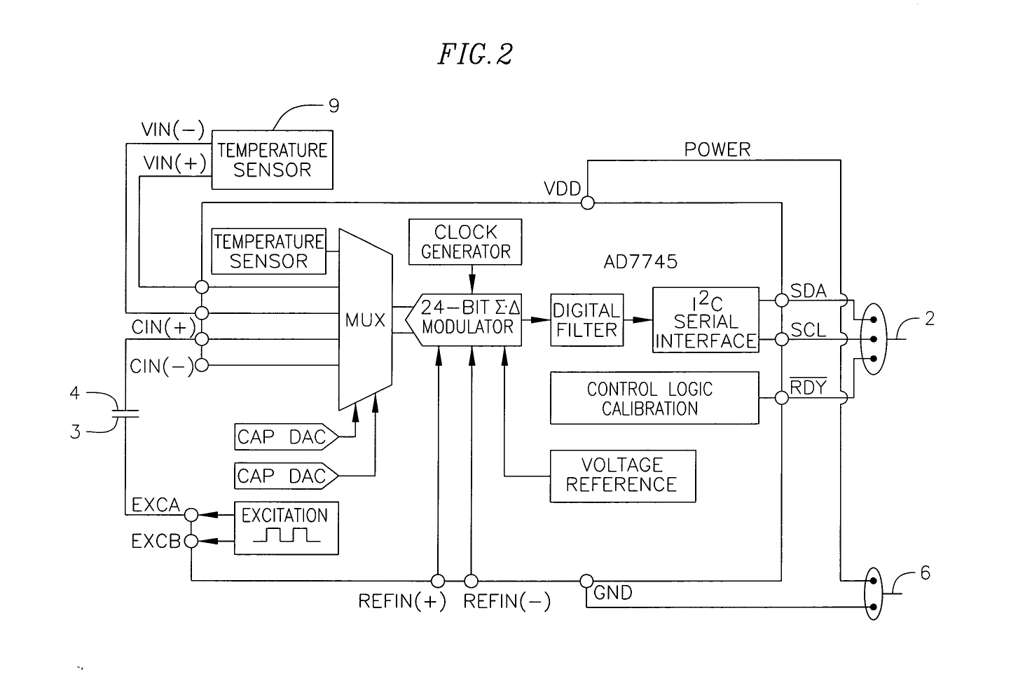

A functional block diagram of a dielectric to digital converter is shown in FIG. 2. The particular capacitance to digital converter or dielectric to digital converter shown in FIG. 2 is an AD7745-24 bit, 1 channel Capacitance to Digital Converter available from Analog Devices (www.analog.com).

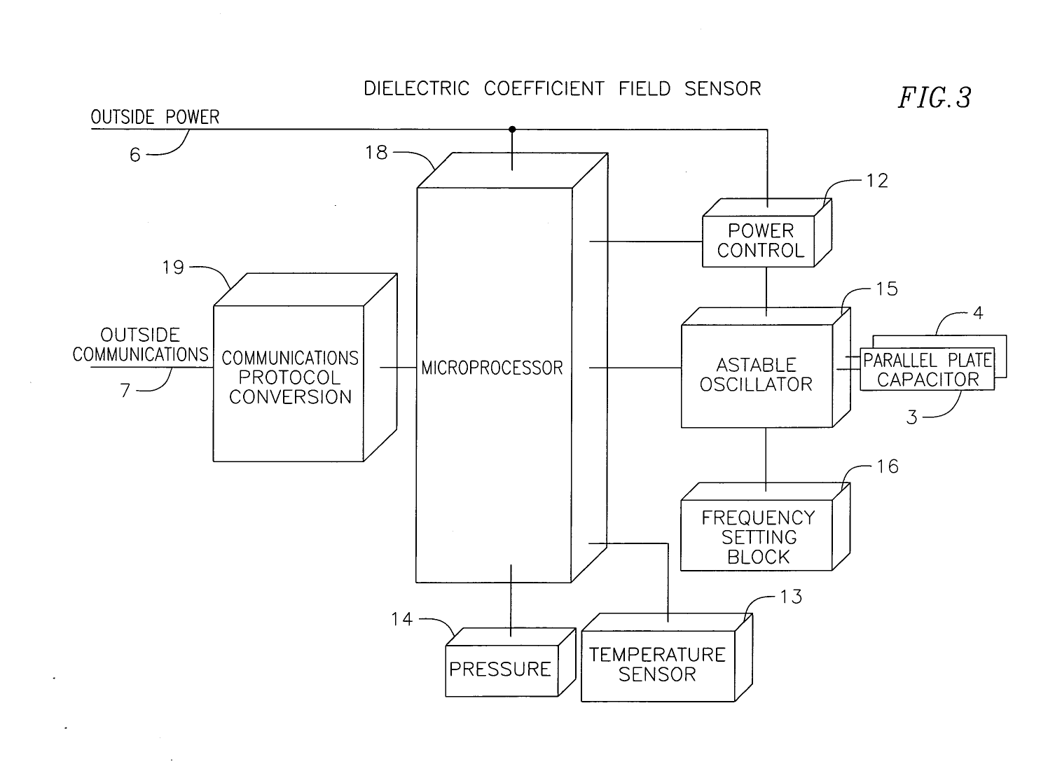

An alternative dielectric to digital (frequency) converter is shown in block form in FIG. 3. This dielectric to digital converter employs an astable oscillator 15 coupled to parallel capacitor plates 3 and 4. The frequency of the astable oscillator is initially set by a variable resistor in a frequency setting block 16. With air between the capacitor plates 3 and 4, the frequency of the oscillator 15 is set at 200 kilohertz. Thereafter, the frequency of the oscillator 15 varies as the dielectric or liquid between the capacitor plates 3 and 4. The variable frequency, at the output of the oscillator 15, is converted to a digital signal representative of the coefficients of the various liquids that pass between the capacitor plates 3 and 4 and applied to a microprocessor 18, functioning as a signal processor. The converted coefficient digital signal from the output of the microprocessor 18 is applied to a communications protocol converter 19 for communication with the selected communication network outside the tank containing the liquids being measured. A temperature sensor 13 is coupled to an input of the microprocessor 18 to monitor the temperature of the liquids that pass between the capacitor plates 3 and 4. Again, the pressure of a single liquid above the capacitive probe may be sensed by a pressure transducer 14 which provides an output that may be used to determine the volume of the single liquid above the pressure transducer 14.

For use in natural gas liquids, the capacitor plates 3 and 4 are 1 centimeter by 8 centimeters in width and length and have a spacing of 1½ centimeters. The size and spacing will be dictated by the environment of use of the capacitive probe 1. Each plate 3 and 4 is coated with an insulating material such as a plastic that will stand up in a corrosive and caustic environment such as is found in liquids from gas wells.

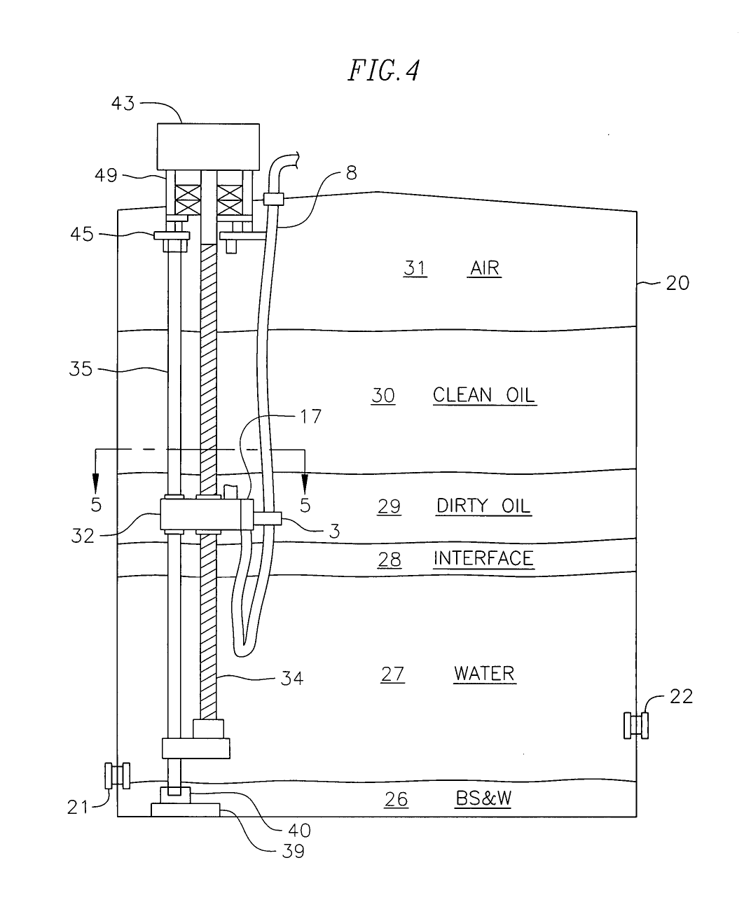

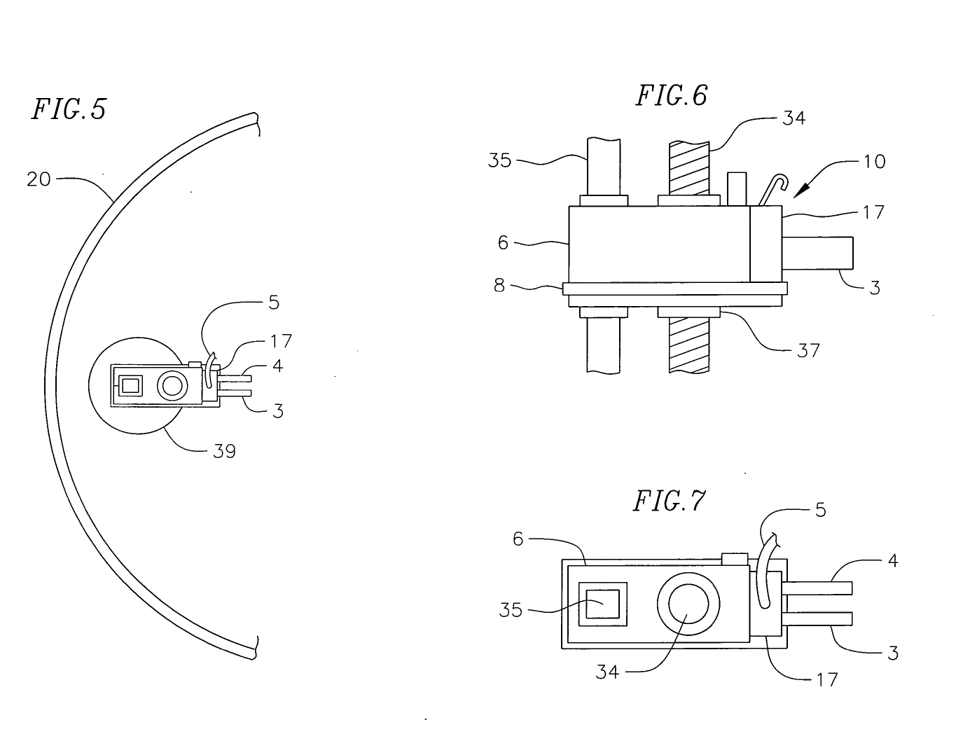

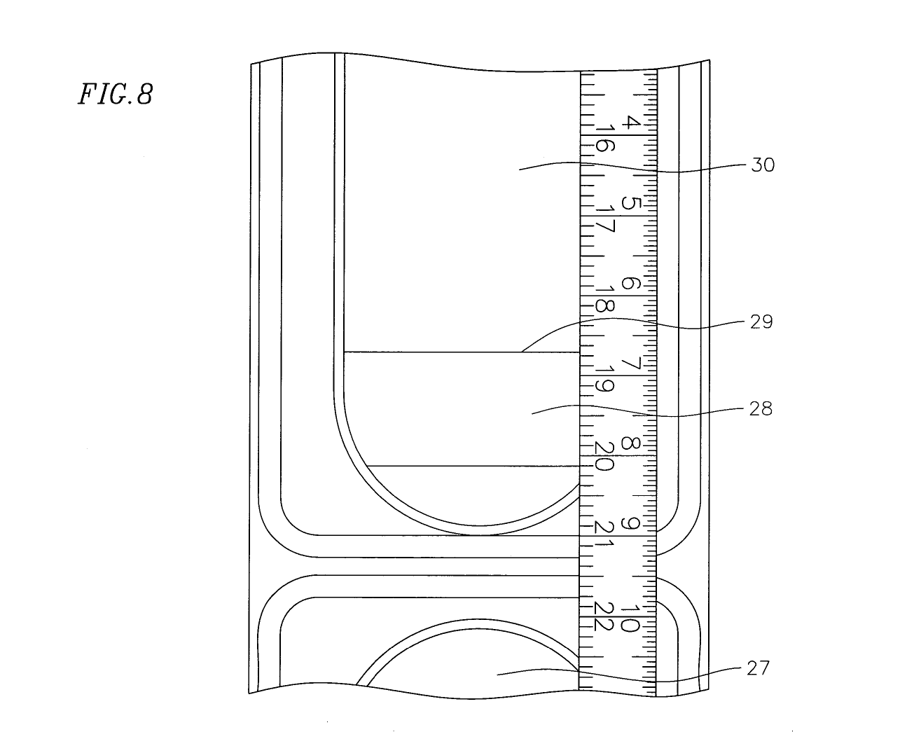

Various components of the capacitive probe 10 shown in FIGS. 1 and 3 may be housed in one housing or separately. For improved stability in operation and reduced interference the dielectric to digital converter 1 of FIGS. 1 and 3 and communication protocol converter 5 or converter 19 are housed together. A housing 17 is shown in FIGS. 4-7 as having a rectangular shape with flat rectangular capacitor plates 3 and 4 extending out to interface with a dielectric between the plates. The housing 17 is constructed of plastic, such as PVC, or other material that withstands the environment of use and includes a inner box for mounting the electronic circuitry of the probe 10.

The capacitive probe may be installed in new tanks (original installation) or in tanks in use (retrofit installation). The tank 20 diagrammatically shown in FIG. 4 represents a 400-barrel or 500-barrel tank that is commonly used to store liquids at gas well sites. Tank 20 has two outlets that are common in such tanks, the lower one 21 being a drain port and a higher one 22 being the load-out port. Typically the drain port 21 is used to remove water and other liquids to bring the lower surface of the clean oil to a point just above the drain port 21. In this way, the lower surface of the oil is below the load-out port 22 to satisfy a requirement that is common in the gas fields; that is, the lower surface of the clean oil must be 8″ below the center of the load-out port 22. Alternatively, the controller 24 may be placed in a box that is placed away from the tank and may be mounted on a pole (not shown).

The capacitive probe 10 for content discrimination may be moved through the liquids in a number of different ways. Tanks that are located at well sites, compressor stations and water plants in gas production are typically of the 400 barrel or 500 barrel size and stand 20 feet tall. For this application, one advantageous way of moving the content discrimination sensor vertically in the tank through the liquids, is by use of a lead screw as shown in FIG. 1. Linear actuators above or inside the tank may be employed to move and position the sensor.

The tank represented in FIG. 4 is a 20 foot tall 400 or 500-barrel tank that is commonly used in gas fields. The liquids in the tank in order of specific gravity from the bottom is: bottom sediment and water (BS&W) 26, water 27, waste oil or interface 28, dirty oil 29, clean oil 30 and air 31 which may also include some gas from the well. The use of the terms waste oil, dirty oil and clean oil in this representative use of the content discrimination sensor are consistent with the definitions set forth in the Onshore Oil and Gas Order No. 4 Measurement of Oil brochure issued by the Bureau of Land Management.

The housing 17 of the probe 10 is attached to a platform or carriage 32 by some means such as a strap 38. The carriage includes a cylindrical hole 33 that is internally threaded to mate with a lead screw 34. In this way the carrier 32 acts as a nut on the lead screw to move the probe vertically as the lead screw 34 is turned. A square pole 35 extends from the top to the bottom of the tank 20 to guide the carrier 32. The internally threaded hole, which functions as the nut on the lead screw 34, may be a separate piece such as a bushing 37 that is inserted in the carrier 32. In this way, the bushing may be made of a different material than the carrier 32. In any event, the material that engages the lead screw 34 should have a surface and consistency that permits easy movement on the lead screw 34. The bushing 37 may advantageously be made of carbon filled PTFE which has a surface that moves easily on the lead screw 34 and provides grounding to avoid static electricity. The support pole 35 is attached at the bottom through a plate 39 and extension 40. Extension 40 has a square opening that corresponds to the square shape of the pole 35 and includes a means for holding the support pole 35 in the plate 39. A drive mechanism 42 on top of the tank 20 is coupled to the lead screw 34 to turn the lead screw at a desired rate. The drive mechanism 42 includes an electric motor 43 and an adaptor 44 in the top of the tank 20 for mounting the drive mechanism 42 and lead screw 34 and support pole 35. The adaptor 44 is threaded at the bottom to receive a support plate 45 that is threaded into the bottom of the adaptor 44 to locate and support the support pole 35. The lead screw 34 is mounted in a pair of thrust bearings 47 and 48 held in place in the adaptor 44 by a shelf at the bottom of adaptor 44 and a nut 49 threaded into the top of the adaptor 44.

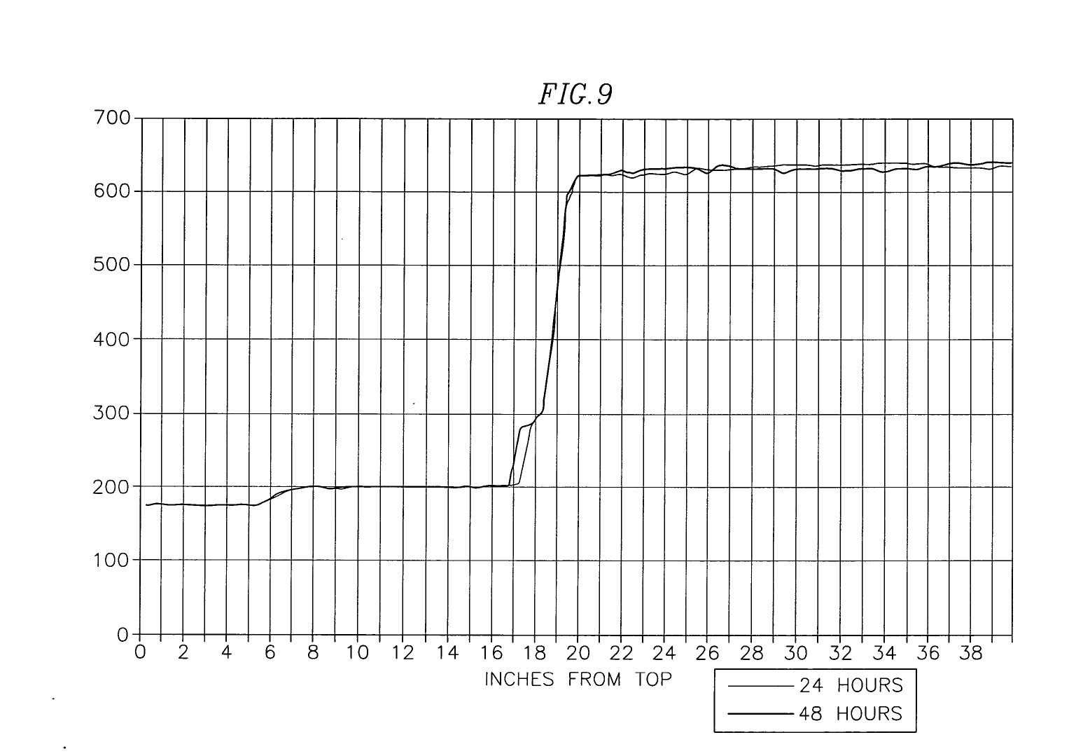

A photograph of a test tank with natural gas liquids is shown in FIG. 8. The photo of FIG. 8 shows liquids from gas wells in a test tank where the liquids have settled over a 48 hour period of time. The liquids have stratified with clean oil 30 on top, dirty oil 29 below the clean oil 30, waste oil or interface 28 below the dirty oil 29 and water 27 below the other liquids.

FIG. 9 is a graph of the output signals from the microprocessor 18 of FIG. 3. The X axis or horizontal axis of the graph is in inches from the top of the test tank (FIG. 8) and corresponds with the tape measure that is shown in the photo of FIG. 8. It is seen that there is a change in dielectric coefficient between 6 and 7 inches on the graph of FIG. 9 where the sensor has moved from air on the left to clean oil; the clean oil having a valve of approximately 200 on the graph of FIG. 9. The next change in coefficient shown on the graph of FIG. 9 is at 17 inches where it is seen from FIG. 8 that the sensor was still in oil. However, this oil area is different from the clean oil 30 above and has been labeled dirty oil 29 because it contains contaminates, such as bacteria, which has changed the coefficient. There is another change at about 18 inches and extends to 20 inches when the sensor passed through the waste oil or interface 28 and entered into the water 27. The water 27 has a valve as shown on the graph of FIG. 9 of approximately 630 to 640. It should be noted that the graph of FIG. 9 is the result of smoothing or averaging the output of the oscillator 15 in microprocessor 18.

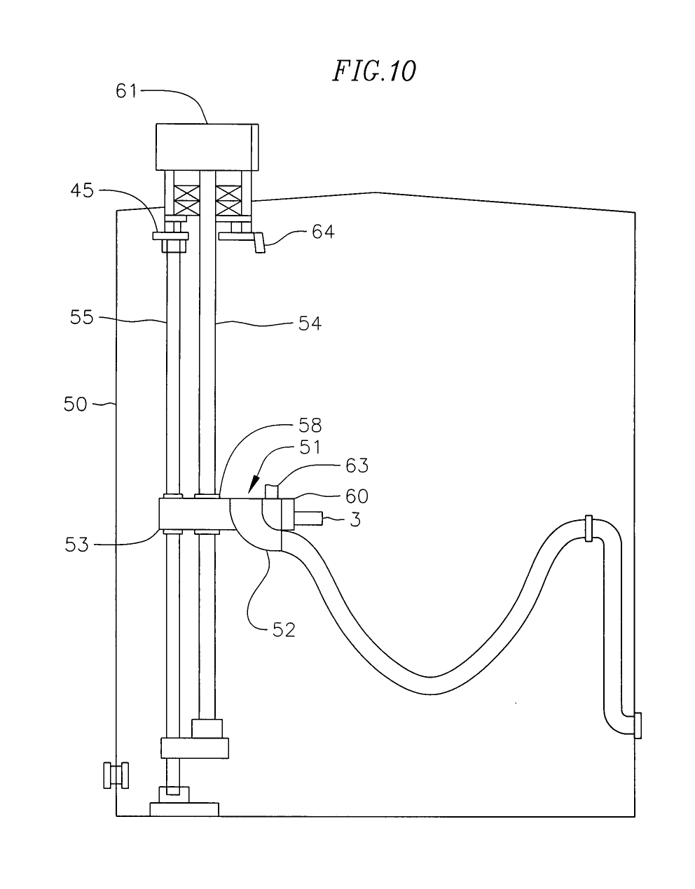

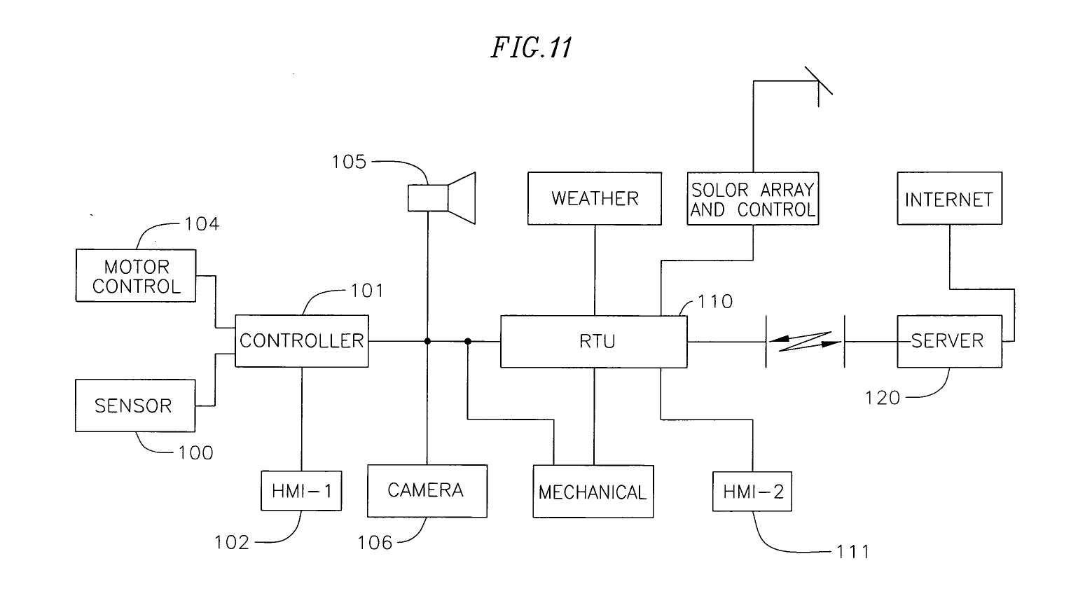

Variable height inlet/outlet orifices have been found to be very useful in storage tanks in gas fields. A number of these variable height inlet/outlet orifices and ways of controlling the position of the orifice are disclosed in the above-referenced PCT Application. One type of variable height inlet/outlet orifice mechanism is shown in FIG. 10. An opening or orifice 51 in an elbow 52 is provided to receive a selected liquid inside a tank 50. The elbow 52 is attached to a carrier 53 that is moved vertically in the tank by a lead screw 54. The carrier 53 slides on and is guided by a support pole 55 that extends from the top to the bottom of the tank 50. The carrier 53 has a bushing 58 that is internally threaded and mates with the threads of the lead screw 54. Thus, when the lead screw 54 turns, the carrier 53 moves vertically to position the orifice 51 at a selected position in the tank 50. A capacitive probe 60 is attached to an extension 59 of the carrier 53 so that the capacitive probe may be moved vertically inside the tank 50 whenever the lead screw 54 is turned or rotated. A drive mechanism outside and on top of the tank 50 turns the lead screw 54 to move the carrier 53 vertically inside the tank 50. The drive mechanism 61, though the lead screw 54 and carrier 53, determine the position of both the sensor 60 and the orifice 51. To survey the contents of the tank 50 and provide a picture of the contents, the carrier 53 is moved from the top of the tank 50 to the bottom of the tank while sensing the dielectric constant of the liquids in the tank. A magnet 63 is attached to and extends above the carrier 53 and is sensed by and triggers a switch 64 attached to the mounting plate or support plate 45 at the top of the tank 50. The sensing of the magnet 63 by the sensor and switch 64 initiates the operation of the capacitive sensor 60 and provides a reference for the position of the sensor. As the lead screw 54 turns and moves the sensor 60 through the liquids, the position of the sensor 64 is referenced to the top of the tank through the switch 64 and magnet 63.

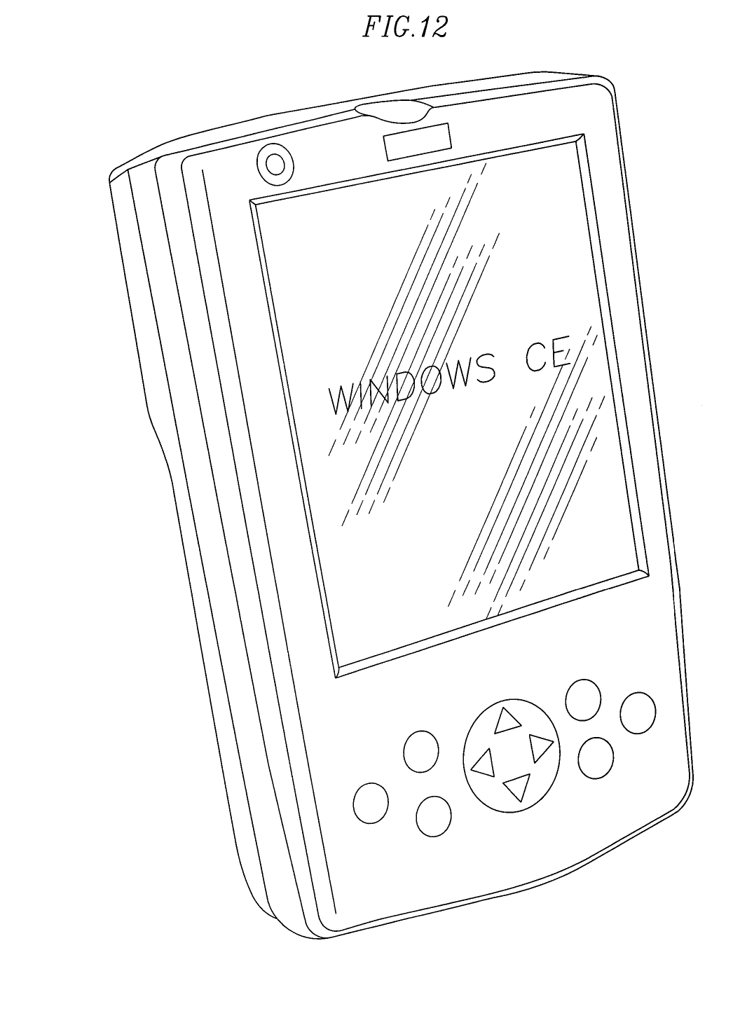

The operation of the capacitive sensor 60 and the moveable orifice 51 controlled by the motor 61 in a system is shown in block form in FIG. 11. The output of the sensor 60 is sent by way of mod bus or some other protocol or some other way of communication to a controller 70. The controller 70 sends the information gathered by the sensor 60 to a communication module or remote terminal unit 71. The remote terminal unit may gather information at the storage tank site such as the weather by an sensor W 72 coupled to the RTU 71. The power to the RTU and the system at the storage tank may be provided by a solar panel 73 and solar array and control 74 coupled to the RTU 71. The RTU 71 controls mechanical devices such as valves 75. The operation of the system may be limited to authorized people only by use of a human machine interface device 76 which requires proper identification and password to operate the system.

A network with one representative system on site for sensing, control and communication is shown in FIG. 11. The on-site system is used with a storage tank, such as those found at well sites, compressor stations, water plants and injection wells associated with gas fields.

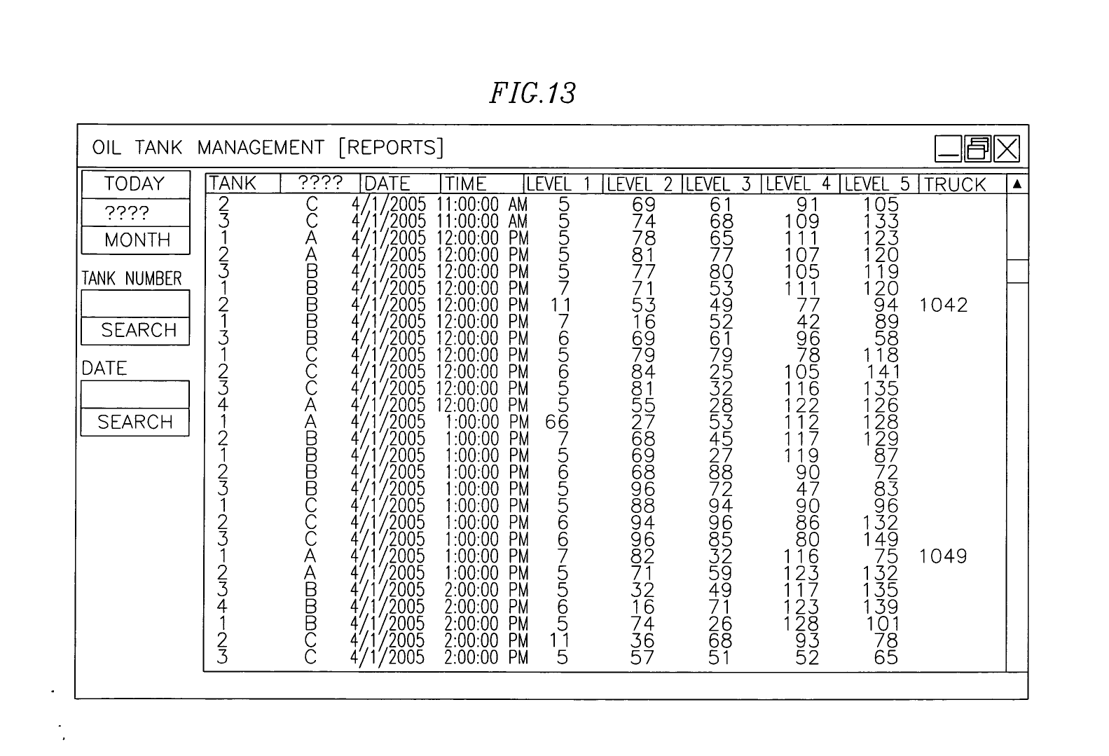

A content discrimination sensor 100 surveys the content of the tank (not shown) and provides the data to a controller 101. The controller 101 may be accessed through a human machine interface 102, such as a PDA (FIG. 12), at the tank site.

The controller 101 controls the mount of the content discrimination sensor 100 through a motor control 104. Motor control 104 may control a separate motor for positioning a variable height inlet/outlet orifice in the tank or the same motor may move both with the content discrimination sensor mounted with an inlet/outlet orifice.

The controller also controls the operation of a horn 105 and a camera 106, which may be provided at the tank site.

The system at the tank site may be controlled from a remote control station with a remote terminal unit (RTU) 110 at the tank site. The on-site management may also be accomplished through the RTU 110 with a human machine interface 111.

A diagram of the front of a programmable, Bluetooth-enabled PDA is shown in FIG. 12. Such a PDA is available from MIG, Palm, HP, and literally dozens of other manufactures and may be used by operators at well sites to interact with the content discrimination sensor system and motorized drive for the variable height inlet/outlet orifice. Bluetooth is a developing, world wide, open, short-range radio specification that defines communication protocols between devices and computers. In other words, the content discrimination sensor may be Bluetooth-enabled to communicate with Bluetooth enabled PDAs. A programmable PDA allows custom application software to be run on the PDA, completing the link between the PDA, the content discrimination sensor, and an optional drive motor to adjust the position of a variable height inlet/outlet orifice.

Application software for the PDA may have the following characteristics:
Image rendering based on digital data from the capacitive console.
Interface controls that allow the operator to designate a stratified layer within a tank.
A Send control that allows the operator to tell the motor to position the inlet/outlet orifice at a certain layer.
For tanks having a ground-level crank, rather than a motor, application software may notify the operator when the orifice has reached the desired location.
Application software may include database capability, automatically logging the operator's identify, the tank being accessed, the time and date, and the specific gravity and quantity of liquid removed. The data may also include the temperature of the liquid and/or the specific gravity. This information may be used for custody transfer and/or inventory control.

A radio transmitter/receiver operating in the 900 MHz range, which is available from MaxStream of Lindon, Utah, may be used to transmit content discrimination data to a central location, such as server 120 of FIG. 11. In some instances, a 900 MHz device may be sufficient. In other instances, however, a repeater, or cellular modem, may be necessary to transmit/receive data.

A graphical interface for a desktop software application that could be used to proactively manage hundreds, or even thousands, of storage tanks throughout a local oil field or a world-wide oilfield is shown in FIG. 13. The software, via wireless radio transmitter technology or a cellular modem, may be set to poll each tank on a regular basis-for example, once per hour-depending on the frequency desired by an operator. Based on the polling data received from each tank, the software may provide a variety of reports not now available to oil and gas companies. Further, the software may provide management assistance in planning daily, weekly, and monthly trucking schedules, thus eliminating significant unnecessary trucking charges that arise from pumpers dispatching trucks for less-than-a-load quantities. The software application may be set to upload critical tank data on a periodic basis to an Internet ISP/Storage facility, such as Center7, of Lindon, Utah. Such entities offer multiple layers of redundant security and data protection to customers that need high-volume data storage/archiving.

Salient features of the desktop software application are as follows:
Pre-set periodic polling or real-time access.
On-demand polling for specific tanks or lists of tanks.
Graphical representation of each tank, showing stratified layers, fill rate, percentage of hydrocarbons to water and other materials, as well as other relevant data specified by the operator.
Historical charts for specific wells showing the well performance over the course of time.
Methodology cost analysis. For example, the software may calculate the profitability of current methodology at a particular well site in relation to optional methodology. For example, at well sites utilizing three-phase heater/treaters, which are expensive to buy and to fuel on a monthly basis, the software may calculate the projected savings and ROI of converting to a variable height inlet/outlet orifice approach.
A daily tasks list, which automatically calculates, based on current volume and fill rates for all tanks, which tanks need which types of liquids drawn off and at what time.
A trucking planner; which would automatically coordinate trucking to a particular part of an oil field for the purpose of eliminating less-than-a-load dispatches. For example, the software, based on recent polling data, determines that tanks at three well sites each have 30 barrels of water that need to be removed. The software, therefore, recommends that a single 100-barrel water truck be dispatched to the region to draw off 30 barrels from each tank. This feature may be used on a daily basis or to forecast trucking requirements weeks and months into the future.
An oil-sales planner, which automatically coordinates possible oil sales on any given day.

For example, based on recent polling data regarding current volumes of light oil in tanks as well as fill rates for those tanks, the software determines that six tanks in a particular geographic region contain a total of 100 barrels of light oil. Assuming these tanks utilize variable height orifices, an oil transport may be dispatched to a region to draw off the light oil from the six tanks.
Data reports with multiple sort keys. For example, an operator may want to generate a list of wells producing the highest ratio of hydrocarbons to water in descending order.

By way of example, and without limitation, the invention may be described as a system for managing stratified liquids in a container, such as a storage tank, comprising a variable height, in-tank content discrimination sensor configured to identify liquid layers of differing dielectric coefficient and to indicate an elevation of the liquid layers, and a motorized drive unit for adjusting the orifice of a liquid management tool to intercept a selected layer.

An external power source and a hand-held computer with a visual read-out that receives feedback from the in-tank content discrimination sensor via a wireless connection may transmit coordinates to the motor drive unit regarding orifice positioning. The hand-held computer automatically turns the motor off when the orifice has reached the optimal position, or alternately, notifies the operator that the orifice is in the optimal position. The hand-held computer includes on-board memory for storing tank management data, for example, which operator removed—added—liquid from or to a stratified layer, the date and time of the operation and the volume of the liquid transported. The hand-held computer functionality may be replaced by other types of computers at remote locations which communicate with the in-tank sensor via various long-distance vehicles, such as radio frequency or microwave.

It is to be understood that the above-referenced arrangements are only illustrative of the application of the principles of the present invention in one or more particular applications. Numerous modifications and alternative arrangements in form, usage and details of implementation can be devised without the exercise of inventive faculty, and without departing from the principles, concepts, and scope of the invention as disclosed herein. Accordingly, it is not intended that the invention be limited, but rather the scope of the invention is to be determined as claimed.

What is claimed is:

1. Apparatus for content discrimination of liquids in a container comprising a sensor for sensing at least one characteristic of a liquid and means for moving the sensor through the liquids.

2. Apparatus in accordance with claim 1 wherein the sensor detects and reads the dielectric constant of a liquid.

3. Apparatus in accordance with claim 1 wherein the characteristic is dielectric constant, temperature, pressure, mass or specified gravity individually or in some combination.

4. Apparatus in accordance with claim 1 further comprising means for determining the position of the sensor in the container.

5. Apparatus in accordance with claim 1 further comprising means for determining the level in the container of at least one transition between liquids having a detectable difference in a common characteristic.

6. Apparatus in accordance with claim 5 wherein the common characteristic is dielectric constant.

7. Apparatus in accordance with claim 1 further comprising a port in a wall of the container, an orifice, and a conduit connected between the orifice and the port.

8. Apparatus in accordance with claim 7 further comprising means for positioning the orifice at a selected position in the container.

9. Apparatus in accordance with claim 7 further comprising means for positioning the orifice at a selected position in the container in response to the output of the sensor.

10. The method of managing liquids in a container comprising the steps of moving a sensor through the liquids in the container and sensing a characteristic of the liquid.

11. The method in accordance with claim 10 wherein the sensed characteristic is dielectric constant, temperature, pressure, mass, or specific gravity individually or in some combination.

12. The method in accordance with claim 10 comprising the further step of determining the height in the container of a transition between liquids having a detectable difference in a common characteristic.

13. The method in accordance with claim 12 wherein the common characteristic is dielectric constant.

14. The method in accordance with claim 12 comprising the further step of positioning an orifice, which has a conduit connected between the orifice and a port of the container, at a selected height in the container.

15. The method in accordance with claim 14 comprising the further step of removing liquid through the orifice, conduit and port.

16. The method in accordance with claim 15 comprising the further step of controlling the removal of liquid at the site of the container.

17. The method in accordance with claim 16 comprising the further step of recording the quantity of liquid removed from the container.

18. The method in accordance with claim 15 comprising the further step of controlling the removal of liquid at a location remote from the site of the container.

19. The method in accordance in with claim 18 comprising the further step of recording the quantity of liquid removed from the container.

20. The method in accordance in with claim 14 wherein the liquids are stratified natural gas liquids and the liquid removed is either condensate and/or clean oil, dirty oil, waste oil or water.

21. The method of managing liquids in a plurality of containers comprising the steps of moving a sensor through the liquids in each container, sensing a characteristic of the liquid in each container and determining the height in each container of a transition between liquids having a detectable difference in a common characteristic.

22. The method in accordance with claim 21 comprising the further step of positioning an orifice, which has a conduit connected between the orifice and a port of the container, at a selected height in each container.

23. The method in accordance with claim 22 comprising the further step of controlling the removal of liquid from each container at a central control station.

24. The method in accordance with claim 23 comprising the further step of recording the removal of liquid from a plurality of containers including the quantity removed and at least one other parameter from the list of container identification, operator, day, time and truck identification.

25. The method in accordance with claim 21 comprising the further step of positioning an orifice, which has a conduit connected between the orifice and a port of the container, at a selected height in each container.

26. The method in accordance with claim 24 comprising the further step of employing the recorded information to manage the transport of liquids to or from each container.

27. The method in accordance with claim 15 comprising the further step of adding a substance to a selected liquid through the port, conduit and orifice.
